SEQUENCE LISTING (36 sequences)

SEQ ID NO: 1 - RNA, 23 nt, homo sapiens
ucuuugguuaucuagcuguauga

SEQ ID NO: 2 - RNA, 22 nt, homo sapiens
uagcagcacguaaauauuggcg

SEQ ID NO: 3 - RNA, 22 nt, homo sapiens
aagcugccaguugaagaacugu

SEQ ID NO: 4 - RNA, 23 nt, homo sapiens
gugccuacugagcugauaucagu

SEQ ID NO: 5 - RNA, 22 nt, homo sapiens
cauugcacuugucucggucuga

SEQ ID NO: 6 - RNA, 22 nt, homo sapiens
aaggagcucacagucuauugag

SEQ ID NO: 7 - RNA, 22 nt, homo sapiens
uguaaacauccucgacuggaag

SEQ ID NO: 8 - RNA, 22 nt, homo sapiens
aaagugcuguucgugcagguag

SEQ ID NO: 9 - RNA, 22 nt, homo sapiens
aacccguagauccgaacuugug

SEQ ID NO: 10 - RNA, 23 nt, homo sapiens
agcagcauuguacagggcuauga

SEQ ID NO: 11 - RNA, 23 nt, homo sapiens
agcagcauuguacagggcuauca

SEQ ID NO: 12 - RNA, 23 nt, homo sapiens
uggagugugacaaugguguuugu

SEQ ID NO: 13 - RNA, 22 nt, homo sapiens
uuaaggcacgcggugaaugcca

SEQ ID NO: 14 - RNA, 21 nt, homo sapiens
cauuauuacuuuugguacgcg

SEQ ID NO: 15 - RNA, 22 nt, homo sapiens
uaacagucuacagccauggucg

SEQ ID NO: 16 - RNA, 23 nt, homo sapiens
acuccauuuguuuugaugaugga

SEQ ID NO: 17 - RNA, 21 nt, homo sapiens
agugguuuuacccuaugguag

SEQ ID NO: 18 - RNA, 20 nt, homo sapiens
cauaaaguagaaagcacuac

SEQ ID NO: 19 - RNA, 20 nt, homo sapiens
cauaaaguagaaagcacuac

SEQ ID NO: 20 - RNA, 22 nt, homo sapiens
ugagaugaagcacuguagcuca

SEQ ID NO: 21 - RNA, 24 nt, homo sapiens
guccaguuuucccaggaaucccuu

SEQ ID NO: 22 - RNA, 22 nt, homo sapiens
ucuggcuccgugucuucacucc

SEQ ID NO: 23 - RNA, 22 nt, homo sapiens
ucagugcaugacagaacuuggg

SEQ ID NO: 24 - RNA, 22 nt, homo sapiens
uagguuauccguguugccuucg

SEQ ID NO: 25 - RNA, 21 nt, homo sapiens
ucgugucuuguguugcagccg

SEQ ID NO: 26 - RNA, 22 nt, homo sapiens
caacggaaucccaaaagcagcu

SEQ ID NO: 27 - RNA, 21 nt, homo sapiens
uagcagcacagaaauauuggc

SEQ ID NO: 28 - RNA, 22 nt, homo sapiens
uccuucauuccaccggagucug

SEQ ID NO: 29 - RNA, 22 nt, homo sapiens
uggaauguaaggaagugugugg

SEQ ID NO: 30 - RNA, 22 nt, homo sapiens
cugugcgugugacagcggcuga

SEQ ID NO: 31 - RNA, 20 nt, homo sapiens
cauaaaguagaaagcacuac

SEQ ID NO: 32 - RNA, 21 nt, homo sapiens
uaaucucagcuggcaacugug

SEQ ID NO: 33 - RNA, 21 nt, homo sapiens
ugauuguccaaacgcaauucu

SEQ ID NO: 34 - RNA, 23 nt, homo sapiens
agcuacauugucugcuggguuuc

SEQ ID NO: 35 - RNA, 21 nt, homo sapiens
ucccugagaccucaaguguga

SEQ ID NO: 36 - RNA, 21 nt, artificial sequence
  synthetic oligonucleotide
ucccugagagcucaaguguga

(19) United States
(12) Patent Application Publication
(10) Pub. No.: US 2006/0292617 A1
(43) Pub. Date: Dec. 28, 2006

(54) Methods and compositions for analysis of microRNA

(75) Inventors: Lori A. Neely, Reading, MA (US); Maria Hackett, Newburyport, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: U.S. Genomics, Inc., Woburn, MA (US)

(21) Appl. No.: 11/474,088

(22) Filed: Jun. 23, 2006

Related U.S. Application Data
(60) Provisional application No. 60/693,334, filed on Jun. 23, 2005.

(51) Int. Cl. C12Q 1/68; C12M 1/34

(52) U.S. Cl. 435/6; 435/287.2; 977/924

(57) ABSTRACT

The invention provides methods and systems for detecting and measuring microRNAs.

### RELATED APPLICATIONS

This application claims priority to U.S. Provisional Application having Ser. No. 60/693,334, and entitled “METHODS AND COMPOSITIONS FOR ANALYSIS OF MICRORNA”, filed on Jun. 23, 2005, the entire contents of which are incorporated by reference herein.

### FIELD OF THE INVENTION

The invention provides methods and compositions for analysis of microRNA, including detection and quantitation.

### BACKGROUND OF THE INVENTION

Short non-coding RNA molecules are potent regulators of gene expression. First discovered in C. elegans (Lee 1993) these highly conserved endogenously expressed ribo-regulators are called microRNAs (miRNAs). miRNAs are short naturally occurring RNAs generally ranging in length from about 7 to about 27 nucleotides.

Only a few hundred miRNAs have been identified. This number is far lower than the expected number of coding sequences in the human genome. However, it is not expected that each coding sequence has its own unique miRNA. This is because miRNAs generally hybridize to RNAs with one or more mismatches. The ability of the miRNA to bind to RNA targets in spite of these apparent mismatches provides the variability necessary to potentially modulate a number of transcripts with a single miRNA.

miRNA therefore can act as regulators of cellular development, differentiation, proliferation and apoptosis. miRNAs can modulate gene expression by either impeding mRNA translation, degrading complementary miRNAs, or targeting genomic DNA for methylation. For example, miRNAs can modulate translation of miRNA transcripts by binding to and thereby making such transcripts susceptible to nucleases that recognize and cleave double stranded RNAs. miRNAs have also been implicated as developmental regulators in mammals in two recent mouse studies characterizing specific miRNAs involved in stem cell differentiation (Houbaviy H B 2003; Chen C Z 2004). Numerous studies have demonstrated miRNAs are critical for cell fate commitment and cell proliferation (Brennecke J 2003) (Zhao Y 2005). Other studies have analyzed the role of miRNAs in cancer (Michael M Z 2003; Calin 2004; He 2005; Johnson S M 2005). miRNAs may play a role in diabetes (Poy M N 2004) and neurodegeneration associated with Fragile X syndrome, spinal muscular atrophy, and early on-set Parkinson's disease (Caudy 2002; Hutvagner 2002; Mouelatos 2002; Dostie 2003). Several miRNAs are virally encoded and expressed in infected cells (e.g., EBV, HPV and HCV).

Analysis of the role of miRNA in these processes, as well as other applications, would be aided by the ability to more accurately and specifically detect and measure miRNA. However, the short nature of the miRNAs makes them difficult to quantify using conventional prior art methods. For example, although Northern blotting has been the “gold standard” for miRNA quantification, this technique is limited in its sensitivity, throughput, and reproducibility. In addition, Northern blotting requires 10-30 micrograms of tissue total RNA and a typical experiment takes 24 to 48 hours to perform with long incubations required for probe hybridization and blot exposure.

There exists a need for methods and systems for detecting and quantitating miRNA, preferably without the need for nucleic acid amplification. Such methods are preferably robust, specific and sufficiently sensitive to abolish the need for amplification.

### SUMMARY OF THE INVENTION

In its broadest sense, the invention provides methods and systems (and corresponding reagents) for detecting and optionally quantitating microRNA (miRNA) in a sample. The method may quantitate all known miRNAs within a complex total RNA sample. It is theoretically unlimited in its degree of multiplexing and offers increased specificity.

In one aspect, the method comprises contacting a template nucleic acid with a miRNA and allowing the template nucleic acid to bind to the miRNA thereby creating a double stranded hybrid with a 5′ template overhang, polymerizing (i.e., synthesizing) a nucleic acid tail to the miRNA wherein the nucleic acid tail is complementary to the 5′ template overhang (or a part thereof) and thereby creating a tailed miRNA, separating the template nucleic acid from the tailed miRNA, contacting a first and a second sequence-specific probe with the tailed miRNA and allowing the first and second sequence-specific probes to bind to the tailed miRNA wherein the first and second sequence-specific probes are complementary to the tailed miRNA, contacting the tailed miRNA to a nucleic acid complementary to the nucleic acid tail and conjugated to a solid support at a defined location (i.e., a capture nucleic acid or a capture probe) and allowing the tailed miRNA to bind to the solid support at the defined location (via binding to the capture nucleic acid), and detecting the level of binding of the tailed miRNA to the solid support based on the presence of the first and second sequence-specific probes at the defined location.

In a related aspect, the method involves contacting one sequence-specific probe with the tailed miRNA and allowing the sequence-specific probe to bind to the tailed miRNA wherein the sequence-specific probe is complementary to the tailed miRNA (preferably within the miRNA specific region), contacting the tailed miRNA to a nucleic acid complementary to the nucleic acid tail and conjugated to a solid support at a defined location (i.e., a capture nucleic acid or a capture probe) and allowing the tailed miRNA to bind to the solid support at the defined location (via binding to the capture nucleic acid), and detecting the level of binding of the tailed miRNA to the solid support based on the presence of the sequence-specific probe at the defined location. In one embodiment, the probe is conjugated to a detectable label. The detectable label may be a fluorophore.

In one embodiment, the first and second sequence-specific probes are conjugated to first and second detectable labels, respectively. The labels are preferably distinct from each other. In some embodiments, the first and second detectable labels are first and second fluorophores.

In one embodiment, the template nucleic acid is about 50% longer than the miRNA. In one embodiment, the miRNA is between 7 and 27 nucleotides in length, and preferably less than 25 nucleotides in length. In another embodiment, the 5′ template overhang is at least 10 bases in length.

In one embodiment, the tailed miRNA is contacted with the first and second sequence-specific probes prior to contact with and binding to the solid support (via the capture nucleic acid). In another embodiment, the tailed miRNA is contacted with the first and second sequence-specific probes after contact with and binding to the solid support (via the capture nucleic acid).

In one embodiment, the template nucleic acid is a DNA. In other embodiments, it may comprise non-naturally occurring elements such as PNAs or LNAs or combinations thereof. In one embodiment, the first and second sequence-specific probes are LNA-DNA chimerae or co-polymers.

In one embodiment, the solid support is a silica chip.

In another embodiment, the method further comprises quantitating a plurality of miRNA. The plurality of miRNA is greater than one and will be limited by the number of unique probe pairs (or unique detectable label pairs) and/or the capacity of the solid support. The upper end of the plurality may be equal to or less than 10000, 3000, 1000, 500, 100, 50, 25, 10, or any integer in between as if explicitly recited herein.

In one embodiment, the defined location on the solid support has a plurality of capture nucleic acids conjugated to it. The plurality in this situation is dependent on the capacity and degree of derivatization of the solid support. Accordingly, the plurality of nucleic acids is at least two and equal to or less than 1000, 750, 500, 250, 100 or 50, in some embodiments.

In one embodiment, the nucleic acid tail is polymerized by a primer extension reaction. In a related embodiment, the primer extension reaction comprises a thermophilic exopolymerase.

In one embodiment, the nucleic acid tail is fluorescent.

In one embodiment, the nucleic acid complementary to the nucleic acid tail (i.e., the capture nucleic acid) is a LNA.

In one embodiment, the nucleic acid complementary to the nucleic acid tail (i.e., the capture nucleic acid) is tethered to the solid support via a 3′ ethylene glycol scaffold.

Various embodiments relate to the various aspects recited herein. Some of these embodiments are recited below and it is to be understood that they apply equally to the various aspects of the invention.

These and other embodiments of the invention will be described in greater detail herein.

Each of the limitations of the invention can encompass various embodiments of the invention. It is therefore anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE FIGURES

FIG. 1 is a schematic of the method for quantitating miRNA as provided herein.

FIG. 2 shows the results of a hybridization reaction in which a DNA oligonucleotide was radiolabeled with P³² and hybridized in solution to the lin-4 miRNA (SEQ ID NO:35) spiked into a complex total RNA sample (2 micrograms of E. coli total RNA).

FIG. 3 shows the results of a hybridization reaction in which a DNA oligonucleotide was radiolabeled with P³² and hybridized in solution to the mutant lin-4 miRNA (SEQ ID NO:36) spiked into a complex total RNA sample (2 micrograms of E. coli total RNA).

FIG. 4 shows the specific extension of a fluorescently labeled DNA tail onto lin-4. Extension reactions utilized Therminator (NEB) with sub-optimal concentrations of nucleotides (200 nM). The reactions were cycled 20 times (90° C. denaturation, 50° C. hybridization, 70° C. extension).

It is to be understood that the Figures are not required for enablement of the invention.

### BRIEF DESCRIPTION OF THE SEQUENCE LISTING

SEQ ID NOs:1-34 are nucleotide sequences of a number of human miRNA, as shown herein.

SEQ ID NO:35 is the nucleotide sequence of a wild type lin-4 miRNA.

SEQ ID NO:36 is the nucleotide sequence of a point mutant lin-4 miRNA.

### DESCRIPTION OF THE INVENTION

The methods of the invention can be used to generate information about miRNA. The information obtained by analyzing a miRNA may include its detection in a sample, determination of the amount or level of the miRNA in a sample and how such amounts vary depending on one or more factors including conditions, timing or the presence of other molecules, determination of the relatedness of more than one miRNA, identification of the size of the miRNA, determination of the proximity or distance between two or more individual units within an miRNA, determination of the order of two or more individual units within an miRNA, and/or identification of the general composition of the miRNA.

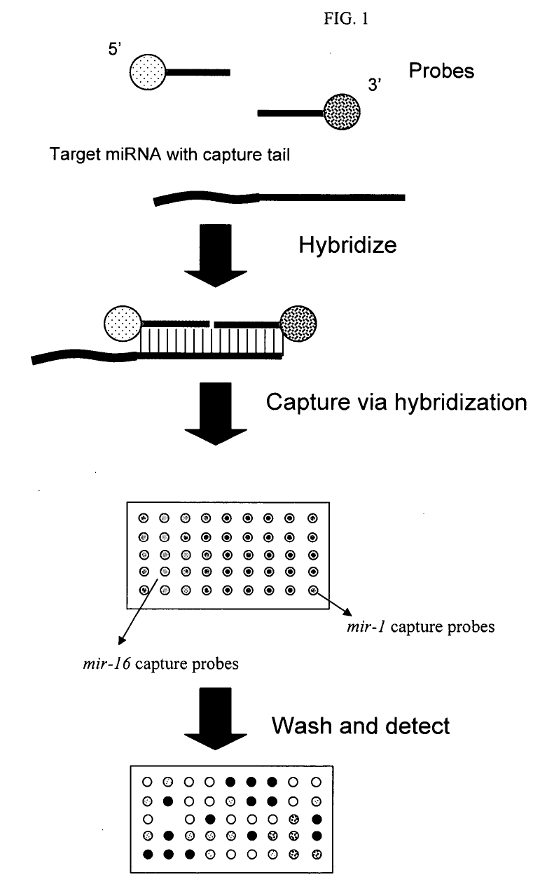

The invention provides a method and system for detecting and quantitating one or more miRNAs simultaneously. The ability to detect more than one miRNA simultaneously is referred to herein as multiplexing capacity. The method of the invention is generally an assay involving the steps of i) hybridization of a template nucleic acid to a miRNA, ii) selective polymerization of a tail onto the end of the hybridized miRNA, iii) hybridization of two spectrally distinctly labeled probes to the tailed miRNA, iv) capture of the labeled tailed miRNAs to a solid surface, and v) measurement of the signal from the labeled tailed miRNA bound to the solid surface. The schematic of the assay is presented in FIG. 1. Each of these steps is discussed in greater detail herein.

The method of one aspect of the invention comprises contacting a template nucleic acid with a miRNA and allowing the template nucleic acid to bind to the miRNA thereby creating a 5′ template overhang. The amount of template used will depend upon the amount of miRNA target. Generally, a 10-50 fold is recommended although higher amounts can be used in some instances.

The template nucleic acid is a nucleic acid comprised of at least two nucleotide sequences. The first sequence is miRNA specific (i.e., it binds to an miRNA target if that target is present in the sample being analyzed). The second sequence is used to generate the tail off of the miRNA “primer” and thus controls the sequence of the tail and ultimately the capture nucleic acids used on the solid supports. This latter sequence may be random, although preferably it is known. Templates that differ in their miRNA specific sequence may also differ in their tail specific sequence, particularly if miRNA identification relies on the location of binding onto the solid support. If miRNA identification relies on the specific probe or probe pairs (and more specifically the signal or coincident signals), then the tail specific sequences may be the same amongst different template nucleic acids.

The template nucleic acid may be comprised of naturally and/or non-naturally occurring elements. For example, it may be a DNA, RNA, PNA, LNA, or a combination thereof. The template exhibits some degree of homology to one or more miRNA. Preferably that level of homology is at least 75%, and includes at least 80%, at least 85%, at least 90%, at least 95%, at least 96%, at least 97%, at least 98%, at least 99% or 100%.

Binding of the template nucleic acid to the miRNA preferably occurs via Watson Crick binding due to the greater sequence specificity it provides. Hybridization of the template to the miRNA is performed under conditions that provide the desired level of stringency and sequence specificity. Those of ordinary skill in the art will be familiar with standard hybridization conditions and manipulation thereof. (See, for example, Maniatis' Handbook of Molecular Biology.) As used herein with respect to two nucleic acids, the terms binding and hybridizing are used interchangeably.

Hybridization of the template to the miRNA results in the formation of a 5′ overhang. As used herein, a 5′ overhang is a single stranded region of the template lying 5′ (along the length of the template) to the double stranded hybrid (or duplex) formed by hybridization of the template to the miRNA. The length of the overhang is dependent on the length of the template and of the miRNA to which it hybridizes, as discussed below.

The template may be longer than the miRNA, but it is not so limited. For example, the template may not hybridize to the entire length of the miRNA, provided that it hybridizes to a sufficiently long region of the miRNA to provide specific hybridization and a stable hybrid (i.e., the template and miRNA hybrid should be sufficiently stable to allow synthesis of the miRNA tail). The length of the template will contribute to this stability, with templates that hybridize to the entire miRNA being more suitable than those that bind to only a region of the miRNA. Thus, in some preferred embodiments, the template is at least 5-10 nucleotides longer than the miRNA to which it is targeted, including at least 15, at least 20, at least 25, at least 50 or more nucleotides longer than the miRNA target. In other embodiments, the length of the template can be at least 25%, at least 50%, at least 75%, at least 100%, or at least 200% of the length of the target miRNA. Binding of the template to the miRNA may also create a 3′ overhang, although no nucleic acid synthesis would be expected to occur from this end of the miRNA.

A plurality of template sequences may be added to a population of miRNA (or a population of RNA containing miRNA). Each of the plurality may contain a random or quasi random sequence in the region intended to bind to a miRNA. That is, the sequences of the target miRNA may not all be known a priori, and the invention can be used to determine those sequences.

The method further involves polymerizing a nucleic acid tail to the miRNA using the miRNA as a primer and the 5′ overhang as the complementary strand (or template). As used herein, polymerization refers to the synthesis of new nucleic acid sequence attached to the miRNA. The nucleic acid tail is therefore complementary to all or part of the 5′ overhang. Creation of the nucleic acid tail provides a way of localizing and potentially identifying the miRNA, as will be discussed below in greater detail.

Polymerization of the nucleic acid tail is accomplished enzymatically using a polymerase enzyme, the miRNA as a primer, the overhang as the template, and free nucleotides. The polymerase enzyme is preferably a DNA polymerase such as DNA polymerase I or the Klenow fragment thereof. The Klenow fragment from E. coli DNA polymerase I possesses polymerase activity and 3′->5′ exonuclease activity but lacks 5′->3′ exonuclease activity associated with DNA polymerase I. Even more preferably, the polymerase enzyme is a thermophilic exopolymerase. Use of a thermophilic exopolymerase allows for reaction cycling without significant loss of polymerase activity. These and other polymerases are known in the art and are commercially available from sources such as New England BioLabs.

In some embodiments, the nucleotides used to synthesize the tail are uniquely labeled and thus the synthesized tail is uniquely labeled. Uniquely labeled, as used herein, means that the synthesized tail can be distinguished from the probes that later hybridize to the miRNA (and optionally from all other probes used in a particular reaction mixture), based on different signal emissions. Examples of suitable detectable labels are provided herein.

The length of the nucleic acid tail is predominately controlled by the length of the 5′ overhang. The nucleic acid tail (and conversely the 5′ overhang) must have a length sufficient to bind to a complementary capture nucleic acid (or capture probe) that is located on a solid support. Thus, the length is preferably at least 6 nucleotides, but is more preferably longer (e.g., 10 nucleotides or longer).

Once the nucleic acid tail is synthesized, the template nucleic acid is physically separated from the tailed miRNA. Physical separation can be accomplished by increasing temperature and/or reducing salt concentration to promote “melting” of the template/miRNA hybrid.

Identification of the miRNA is accomplished by binding one or more probes (e.g., first and second) sequence-specific probes to the tailed miRNA. The probes may bind to the miRNA itself or to its tail region, or to a combination thereof. For example, if the tail is sufficiently long, one probe may bind to it (or to a region of it). More commonly, both sequence-specific probes will bind to the miRNA sequence itself. Preferably the probes are bound to the tailed miRNA (regardless of the binding position) under stringent hybridization conditions, as discussed herein. If a combination of probes is used, then the combination must be capable of uniquely identifying the tailed miRNA. The probes may be comprised of DNA, RNA, PNA, LNA or a combination thereof (e.g., a LNA-DNA chimerae). Sequence-specific probes are discussed in greater detail herein.

A plurality of probes may be used and such a plurality may be synthesized using known or random sequences.

The tailed miRNA is positioned on a solid support by hybridizing it to a capture nucleic acid (or capture probe) that is complementary to the nucleic acid tail including a part thereof. The capture nucleic acid is conjugated to the solid support using techniques known in the art. As an example, the capture nucleic acid may be tethered to the solid support via a 3′ ethylene glycol scaffold. (Matsuya et al. Anal Chem. 2003 Nov. 15; 75(22):6124-32.) Preferably the capture nucleic acid is positioned on a solid support in a particular manner. For example, a solid support may be divided into a grid, each square of the grid having one or more capture nucleic acids of a particular sequence conjugated to it. The number of capture nucleic acids that can be conjugated to a square in the grid will depend on a number of factors such as the size of the square, the conjugation technique used, the length of the capture nucleic acid, etc. In some instances, the number of capture nucleic acids may be in the tens, the hundreds, or even the thousands. Each square may contain capture nucleic acids of a particular known sequence. Thus the location of the square can be representative of the particular miRNA being analyzed. The solid support is then scanned using a detection system such as Trilogy™ for squares occupied by one or more sequence-specific probes. Presence of two probes (or two signals) at a given location is indicative of the presence of a miRNA. The amount of dual signals in a defined location is representative of the amount of a particular miRNA captured (and thus the amount of that miRNA in the tested sample). The method can be used to determine the presence of any number of miRNA, including but not limited to up to 5, 10, 25, 50, 100, 300, 100, 3000, or more.

It is to be understood that although the solid support is described herein as having a grid and therefore being divided into squares, the invention is not so limited. It is only necessary that the locations on the solid support be defined. The locations may be referred to, for example, by co-ordinates or by x-y distances relative to a reference spot on the support (e.g., a corner of the solid support).

It is also to be understood that binding of the sequence-specific probes to the tailed miRNA may occur before or after binding of the tailed miRNA to the capture nucleic acid on the solid support. Therefore, in some embodiments, the tailed miRNA is hybridized to the solid support following which detectably labeled probes are added to the solid support. In this way, smaller amounts of probes are necessary since the hybridization volume is small.

Single miRNAs are detected using one or more probes that are specific to the miRNA (i.e., miRNA-specific probes, as discussed herein). A sample may be tested for the presence of miRNA by contacting it with one or more miRNA-specific probes for a time and under conditions that allow for binding of the probe to the miRNA if it is present. Excess probe amounts may be used to ensure that all binding sites are occupied.

If more than one probe is used, such probes are preferably chosen so that they bind to different regions of the miRNA, and therefore cannot compete with each other for binding to the miRNA. Similarly, probes are labeled with distinguishable detectable labels (i.e., the detectable label on the first probe is distinct from that on the second probe). Once the probes are allowed to bind to the miRNA (if it is present in the sample), the sample is analyzed for coincident emission signals (i.e., a distinct and detectable signal from each detectable label). For example, a miRNA bound by two probes is manifest as overlapping emission signals from the bound probes. This detection is accomplished using a single molecule detection or analysis system. A single molecule detection or analysis system is a system capable of detecting and analyzing individual, preferably intact, molecules.

The method is particularly suited to detecting miRNA in a rare or small sample (e.g., a nanoliter volume sample) or in a sample where miRNA concentration is low. The invention allows more than one and preferably several different miRNA to be detected simultaneously, thereby conserving sample. In other words, the method is capable of a high degree of multiplexing. For example, the degree of multiplexing may be 2 (i.e., 2 miRNA can be detected in a single analysis), 3, 4, 5, 6, 7, 8, 9, 10, at least 20, at least 50, at least 100, at least 200, at least 300, at least 400, at least 500, or higher. In one embodiment, each miRNA is detected using a particular probe pair where preferably each member of the probe pair is specific to the miRNA (or at a minimum, one member of the pair is specific to the miRNA) and each probe used in an analysis is labeled with a distinguishable label. Thus, a plurality of miRNA may be detected and analyzed. As used herein, a plurality is an amount greater than two but less than infinity. A plurality is sometimes less than a million, less than a thousand, less than a hundred, or less than ten.

The methods of the invention can be used to determine amount or relative concentration of an miRNA species in a sample. To determine either, the data from a test sample (i.e., a sample having unknown miRNA amount or concentration) are compared to data from one or more control samples (i.e., samples having known miRNA amount or concentration). Generally, a series of control samples are analyzed in order to generate a standard curve and the data from the test sample is plotted against the standard curve to arrive at an amount or concentration.

miRNA Targets

The sequences of numerous miRNA are known and publicly available. Accordingly, synthesis of miRNA-specific probes is within the ordinary skill in the art based on this information. miRNA sequences can be accessed at for example the website of the miRNA Registry of the Sanger Institute (Wellcome Trust), or the website of Ambion, Inc.

For example, some miRNA sequences are as follows:

| | | Accession | SEQ ID |
|---|---|---|---|
| miRNA | Sequence | Number | NO: |
| human | UCUUUGGUUAUCUAGCUGUAUGA | MI0000466 | 1 |
| mir-9 | | | |
| human | UAGCAGCACGUAAAUAUUGGCG | MI0000738 | 2 |
| mir-16 | | | |
| human | AAGCUGCCAGGUGAAGAACUGU | MI0000078 | 3 |
| mir-22 | | | |
| human | GUGCCUACUGAGCUGAUAUCAGU | MI0000080 | 4 |
| mir-24 | | | |
| human | CAUUGCACUUGUCUCGGUCUGA | MI0000082 | 5 |
| mir-25 | | | |
| human | AAGGAGCUCACAGUCUAUUGAG | MI0000086 | 6 |
| mir-28 | | | |
| human | UGUAAACAUCCUCGACUGGAAG | MI0000088 | 7 |
| mir-30a | | | |
| human | AAAGUGCUGUUCGUGCAGGUAG | MI0000095 | 8 |
| mir-93 | | | |
| human | AACCCGUAGAUCCGAACUUGUG | MI0000102 | 9 |
| mir-100 | | | |
| human | AGCAGCAUUGUACAGGGCUAUGA | MI0000109 | 10 |
| mir-103 | | | |
| human | AGCAGCAUUGUACAGGGCUAUCA | MI0000114 | 11 |
| mir-107 | | | |
| human | UGGAGUGUGACAAUGGUGUUUGU | MI0000442 | 12 |
| mir-122a | | | |
| human | UUAAGGCACGCGGUGAAUGCCA | MI0000443 | 13 |
| mir-124a | | | |
| human | CAUUAUUACUUUUGGUACGCG | MI0000471 | 14 |
| mir-126 | | | |
| human | UAACAGUCUACAGCCAUGGUCG | MI0000449 | 15 |
| mir-132 | | | |
| human | ACUCCAUUUGUUUUGAUGAUGGA | MI0000475 | 16 |
| mir-136 | | | |
| human | AGUGGUUUUACCCUAUGGUAG | MI0000456 | 17 |
| mir-140 | | | |
| human | CAUAAAGUAGAAAGCACUAC | MI0000458 | 18 |
| mir-141 | | | |
| human | CAUAAAGUAGAAAGCACUAC | MI0000458 | 19 |
| mir-142 | | | |
| human | UGAGAUGAAGCACUGUAGCUCA | MI0000459 | 20 |
| mir-143 | | | |
| human | GUCCAGUUUUCCCAGGAAUCCCUU | MI0000461 | 21 |
| mir-145 | | | |
| human | UCUGGCUCCGUGUCUUCACUCC | MI0000478 | 22 |
| mir-149 | | | |
| human | UCAGUGCAUGACAGAACUUGGG | MI0000462 | 23 |
| mir-152 | | | |
| human | UAGGUUAUCCGUGUUGCCUUCG | MI0000480 | 24 |
| mir-154 | | | |
| human | UCGUGUCUUGUGUUGCAGCCG | MI0000274 | 25 |
| mir-187 | | | |
| human | CAACGGAAUCCCAAAAGCAGCU | MI0000465 | 26 |
| mir-191 | | | |
| human | UAGCAGCACAGAAAUAUUGGC | MI0000489 | 27 |
| mir-195 | | | |
| human | UCCUUCAUUCCACCGGAGUCUG | MI0000285 | 28 |
| mir-205 | | | |
| human | UGGAAUGUAAGGAAGUGUGUGG | MI0000490 | 29 |
| mir-206 | | | |
| human | CUGUGCGUGUGACAGCGGCUGA | MI0000286 | 30 |
| mir-210 | | | |
| human | CAUAAAGUAGAAAGCACUAC | MI0000458 | 31 |
| mir-213 | | | |
| human | UAAUCUCAGCUGGCAACUGUG | MI0000292 | 32 |
| mir-216 | | | |
| human | UGAUUGUCCAAACGCAAUUCU | MI0000296 | 33 |
| mir-219 | | | |
| human | AGCUACAUUGUCUGCUGGGUUUC | MI0000298 | 34 |
| mir-221 | | | |

RNA Samples

Harvest and isolation of total RNA is known in the art and reference can be made to standard RNA isolation protocols. (See, for example, Maniatis' Handbook of Molecular Biology.) The method does not require that miRNA be enriched from a standard RNA preparation, although if desired the miRNA may be enriched using a YM-100 column.

miRNA may be harvested from a biological sample such as a tissue or a biological fluid. The term “tissue” as used herein refers to both localized and disseminated cell populations including. but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art. In some embodiments, the miRNA are harvested from one or few cells.

The methods of the invention may be performed in the absence of prior nucleic acid amplification in vitro. Preferably, the miRNA is directly harvested and isolated from a biological sample (such as a tissue or a cell culture), without its amplification. Such miRNA are referred to as “non in vitro amplified nucleic acids”. As used herein, a “non in vitro amplified nucleic acid” refers to a nucleic acid that has not been amplified in vitro using techniques such as polymerase chain reaction or recombinant DNA methods.

A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

miRNA to be detected and optionally quantitated are referred to as target miRNA or target nucleic acids.

Sample Manipulation

Although the tailed miRNA may be linearized or stretched prior to analysis, this is not necessary since the detection system is capable of analyzing both stretched and condensed forms. This is particularly the case with coincident events since these events simply require the presence of at least two labels, but are not necessarily dependent upon the relative positioning of the labels (provided however that if they are being detected using FRET, they are sufficiently proximal to each other to enable energy transfer).

As used herein, stretching of the miRNA means that it is provided in a substantially linear, extended (e.g., denatured) form rather than a compacted, coiled and/or folded (e.g., secondary) form. Stretching the miRNA prior to analysis may be accomplished using particular configurations of, for example, a single molecule detection system, in order to maintain the linear form. These configurations are not required if the target can be analyzed in a compacted form.

Coincidence Binding and Detection

Coincident binding refers to the binding of two or more probes on a single molecule or complex. Coincident binding of two or more probes is used as an indicator of the molecule or complex of interest. It is also useful in discriminating against noise in the system and therefore increases the sensitivity and specificity of the system. Coincident binding may take many forms including but not limited to a color coincident event, whereby for example two colors corresponding to a first and a second detectable label are detected. Coincident binding may also be manifest as the proximal binding of a first detectable label that is a FRET donor fluorophore and a second detectable label that is a FRET acceptor fluorophore. In this latter embodiment, a positive signal is a signal from the FRET acceptor fluorophore upon laser excitation of the FRET donor fluorophore.

Some of the methods provided herein involve the ability to detect single molecules based on the temporally coincident detection of detectable labels specific to the miRNA being analyzed. As used herein, coincident detection refers to the detection of an emission signal from more than one detectable label in a given period of time. Generally, the period of time is short, approximating the period of time necessary to analyze a single molecule. This time period may be on the order of a millisecond. Coincident detection may be manifest as emission signals that overlap partially or completely as a function of time. The co-existence of the emission signals in a given time frame may indicate that two non-interacting molecules, each individually and distinguishably labeled, are present in the interrogation spot at the same time. An example would be the simultaneous presence of two unbound but detectably and distinguishably labeled probes in the interrogation spot. However, because the spot volume is so small (and the corresponding analysis time is so short), the likelihood of this happening is small. Rather it is more likely that if two probes are present in the interrogation spot simultaneously, this is due to the binding of both probes to a single molecule passing through the spot. In some embodiments, signals from samples containing labeled probes but lacking miRNA targets are determined and subtracted from signals from samples containing both probes and targets.

The coincident detection methods of the invention involve the simultaneous detection of more than one emission signal. The number of emission signals that are coincident will depend on the number of distinguishable detectable labels available, the number of probes available, the number of components being detected, the nature of the detection system being used, etc. Generally, at least two emission signals are being detected. In some embodiments, three emission signals are being detected. However, the invention is not so limited. Thus, where multiple components are being detected in a single analysis, 4, 5, 6, 7, 8, 9, 10 or more emission signals can be detected simultaneously.

Coincident detection analysis is able to detect single molecules at very low concentrations. For example, as discussed herein, low femtomolar concentrations can be detected using a two or three emission signal approach. In addition, the analysis demonstrates a dynamic range of greater than four orders of magnitude. A two emission signal approach is also able to detect single molecules such as single proteins at levels below 1 ng/ml.

Probes

A probe is a molecule that specifically binds to a target of interest. The nature of the probe will depend upon the application and may also depend upon the nature of the target. Specific binding, as used herein, means the probe demonstrates greater affinity for its target than for other molecules (e.g., based on the sequence or structure of the target). The probe may bind to other molecules, but preferably such binding is at or near background levels. For example, it may have at least 2-fold, 5-fold, 10-fold or higher affinity for the desired target than for another molecule. Probes with the greatest differential affinity are preferred in most embodiments, although they may not be those with the greatest affinity for the target.

Probes can be virtually any compound that binds to a target with sufficient specificity. Examples include nucleic acids that bind to complementary nucleic acid targets via Watson-Crick and/or Hoogsteen binding (as discussed herein), aptamers that bind to nucleic acid targets due to structure rather than complementarity of sequence of the target, antibodies, etc. It is to be understood that although many of the exemplifications provided herein relate to nucleic acid probes, the invention is not so limited and other probes are envisioned.

“Sequence-specific” when used in the context of a probe for a tailed miRNA means that the probe recognizes a particular linear arrangement of nucleotides or derivatives thereof. In preferred embodiments, the sequence-specific probe is itself composed of nucleic acid elements such as DNA, RNA, PNA and LNA elements or combinations thereof (as discussed herein). In preferred embodiments, the linear arrangement includes contiguous nucleotides or derivatives thereof that each binds to a corresponding complementary nucleotide in the probe. In some embodiments, however, the sequence may not be contiguous as there may be one, two, or more nucleotides that do not have corresponding complementary residues on the probe, and vice versa.

Any molecule that is capable of recognizing a nucleic acid with structural or sequence specificity can be used as a sequence-specific probe. In most instances, such probes will be nucleic acids themselves and will form at least a Watson-Crick bond with the tailed miRNA. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. In some embodiments, the nucleic acid probes can form both Watson-Crick and Hoogsteen bonds with the tailed miRNA. Bis PNA probes, for instance, are capable of both Watson-Crick and Hoogsteen binding to a nucleic acid.

The length of the probe can also determine the specificity of binding. The energetic cost of a single mismatch between the probe and its target is relatively higher for shorter sequences than for longer ones. Therefore, hybridization of smaller nucleic acid probes is more specific than is hybridization of longer nucleic acid probes to the same target because the longer probes can embrace mismatches and still continue to bind to the target. One potential limitation to the use of shorter probes however is their inherently lower stability at a given temperature and salt concentration. One way of avoiding this latter limitation involves the use of bis PNA probes which bind shorter sequences with sufficient hybrid stability.

Notwithstanding these provisos, the nucleic acid probes of the invention can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 11 nucleotides, at least 12 nucleotides, at least 13 nucleotides, at least 14 nucleotides, at least 15 nucleotides, at least 20 nucleotides, or at least 25 nucleotides, or more, in length.

The length of the probe may also be represented as a proportion of the length of the miRNA to which it binds specifically. For example, the probe length may be at least 10%, at least 20%, at least 30%, at least 40%, or at least 50% the length of its target miRNA, or longer.

It should be understood that not all residues of the probe need to hybridize to complementary residues in the nucleic acid target, although this is preferred. For example, the probe may be 50 residues in length, yet only 45 of those residues hybridize to the nucleic acid target. Preferably, the residues that hybridize are contiguous with each other.

The probes are preferably single-stranded, but they are not so limited. For example, when the probe is a bis PNA it can adopt a secondary structure with the nucleic acid target (e.g., the miRNA) resulting in a triple helix conformation, with one region of the bis PNA clamp forming Hoogsteen bonds with the backbone of the tailed miRNA and another region of the bis PNA clamp forming Watson-Crick bonds with the nucleotide bases of the tailed miRNA.

The nucleic acid probe hybridizes to a complementary sequence within the tailed miRNA. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

Nucleic Acids and Derivatives Thereof

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine. The nucleic acids may be single- or double-stranded. Nucleic acids can be obtained from natural sources, or can be synthesized using a nucleic acid synthesizer.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid tails, nucleic acid probes and capture nucleic acids.

In some embodiments, the invention embraces nucleic acid derivatives in nucleic acid tails, nucleic acid probes and/or capture nucleic acids. As used herein, a “nucleic acid derivative” is a non-naturally occurring nucleic acid or a unit thereof. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acid derivatives may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

Nucleic acid probes and capture nucleic acids can be stabilized in part by the use of backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, nucleic acid probes and/or capture nucleic acids may include a peptide nucleic acid (PNA), a bis PNA clamp, a pseudocomplementary PNA, a locked nucleic acid (LNA), DNA, RNA, or co-nucleic acids of the above such as DNA-LNA co-nucleic acids (as described in co-pending U.S. patent application having Ser. No. 10/421,644 and publication number US 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

In some important embodiments, the nucleic acid probe is a LNA/DNA chimeric probe. LNA content may vary from more than 0% to less than 100%, and may include at least 5%, at least 10%, at least 20%, at least 30%, at least 40%, at least 50%, at least 60%, at least 70%, at least 80%, at least 90%, at least 95%, or at least 99%. In some embodiments, 10- or 11-mer probes may contain on average about 3-4 LNAs, for example.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA- or RNA-based probes.

PNAs are synthesized from monomers connected by a peptide bond (Nielsen, P. E. et al. Peptide Nucleic Acids Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids, Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bis PNA and pseudocomplementary PNA (pcPNA).

The structure of PNA/DNA complex depends on the particular PNA and its sequence. Single stranded PNA (ssPNA) binds to single-stranded DNA (ssDNA) preferably in anti-parallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

BisPNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bis PNA/DNA complex is still stable as it forms a hybrid with twice as many (e.g., a 16 bp) base pairings overall. The bis PNA structure further increases specificity of their binding. As an example, binding to an 8 bp site with a probe having a single base mismatch results in a total of 14 bp rather than 16 bp.

Pseudocomplementary PNA (pcPNA) (Izvolsky, K. I. et al., Biochemistry 10908-10913 (2000)) involves two single-stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and SU are still capable of hybridization with T and A respectively, their self-hybridization is sterically prohibited.

Locked nucleic acids (LNA) are modified RNA nucleotides. (See, for example, Braasch and Corey, Chem. Biol., 2001, 8(1):1-7.) LNAs form hybrids with DNA which are at least as stable as PNA/DNA hybrids. Therefore, LNA can be used just as PNA molecules would be. LNA binding efficiency can be increased in some embodiments by adding positive charges to it.

Commercial nucleic acid synthesizers and standard phosphoramidite chemistry are used to make LNAs. Therefore, production of mixed LNA/DNA sequences is as simple as that of mixed PNA/peptide sequences. Naturally, most of biochemical approaches for nucleic acid conjugations are applicable to LNA/DNA constructs.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as O-linkers), amino acids such as lysine (particularly useful if positive charges are desired in the PNA), and the like. Various PNA modifications are known and probes incorporating such modifications are commercially available from sources such as Boston Probes, Inc.

Labeling of Sequence-Specific Probes

The probes, and in some instances the miRNA tails, are detectably labeled (i.e., they comprise a detectable label). A detectable label is a moiety, the presence of which can be ascertained directly or indirectly. Generally, detection of the label involves the creation of a detectable signal such as for example an emission of energy. The label may be of a chemical, lipid, peptide or nucleic acid nature although it is not so limited. The nature of label used will depend on a variety of factors, including the nature of the analysis being conducted, the type of the energy source and detector used. The label should be sterically and chemically compatible with the constituents to which it is bound.

The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.).

There are several known methods of direct chemical labeling of DNA. (Hermanson, G. T., Bioconjugate Techniques, Academic Press, Inc., San Diego, 1996; Roget et al., 1989; Proudnikov and Mirabekov, Nucleic Acid Research, 24:4535-4532, 1996.) One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

It is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl)aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl)aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like. Antibody fragments include Fab, F(ab)₂, Fd and antibody fragments which include a CDR3 region.

In some embodiments, the first and second sequence-specific probes may be labeled with fluorophores that form a fluorescence resonance energy transfer (FRET) pair. In this case, one excitation wavelength is used to excite fluorescence of donor fluorophores. A portion of the energy absorbed by the donors can be transferred to acceptor fluorophores if they are close enough spatially to the donor molecules (i.e., the distance between them must approximate or be less than the Forster radius or the energy transfer radius). Once the acceptor fluorophore absorbs the energy, it in turn fluoresces in its characteristic emission wavelength. Since energy transfer is possible only when the acceptor and donor are located in close proximity, acceptor fluorescence is unlikely if both probes are not bound to the same miRNA. Acceptor fluorescence therefore can be used to determine presence of miRNA.

It is to be understood however that if a FRET fluorophore pair is used, coincident binding of the pair to a single target is detected by the presence or absence of a signal rather than a coincident detection of two signals.

A FRET fluorophore pair is two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TMR (Tamra). Examples of acceptors include Cy5, Alexa 594, Alexa 647 and Oyster 656. Cy5 can work as a donor with Cy3, TMR or Alexa 546, as an example. FRET should be possible with any fluorophore pair having fluorescence maxima spaced at 50-100 nm from each other. The FRET embodiment can be coupled with another label on the target miRNA such as a backbone label, as discussed below.

The miRNA may be additionally labeled with a backbone label. These labels generally label nucleic acids in a sequence non-specific manner. In these embodiments, the miRNA may be detected by the coincident signals from the backbone label and one or more of the bound probes. Examples of backbone labels (or stains) include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Therefore, some embodiments of the invention embrace three color coincidence. In these embodiments, single or multiple lasers may be used. For example, three different lasers may be used for excitation at the following wavelengths: 488 nm (blue), 532 nm (green), and 633 nm (red). These lasers excite fluorescence of Alexa 488, TMR (tetramethylrhodamine), and TOTO-3 fluorophores, respectively. Fluorescence from all these fluorophores can be detected independently. As an example of fluorescence strategy, one sequence-specific probe may be labeled with Alexa 488 fluorophore, a second sequence-specific probe may be labeled with TMR, and the miRNA backbone may be labeled with TOTO-3. TOTO-3 is an intercalating dye that non-specifically stains nucleic acids in a length-proportional manner. Another suitable set of fluorophores that can be used is the combination of POPO-1, TMR and Alexa 647 (or Cy5) which are excited by 442, 532 and 633 nm lasers respectively.

Conjugation, Linkers and Spacers

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Non-covalent conjugation includes hydrophobic interactions, ionic interactions, high affinity interactions such as biotin-avidin and biotin-streptavidin complexation and other affinity interactions. Such means and methods of attachment are known to those of ordinary skill in the art. Conjugation can be performed using standard techniques common to those of ordinary skill in the art.

The various components described herein can be conjugated by any mechanism known in the art. For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

Linkers and/or spacers may be used in some instances. Linkers can be any of a variety of molecules, preferably nonactive, such as nucleotides or multiple nucleotides, straight or even branched saturated or unsaturated carbon chains of C₁-C₃₀, phospholipids, amino acids, and in particular glycine, and the like, whether naturally occurring or synthetic. Additional linkers include alkyl and alkenyl carbonates, carbamates, and carbamides. These are all related and may add polar functionality to the linkers such as the C₁-C₃₀ previously mentioned. As used herein, the terms linker and spacer are used interchangeably.

A wide variety of spacers can be used, many of which are commercially available, for example, from sources such as Boston Probes, Inc. (now Applied Biosystems). Spacers are not limited to organic spacers, and rather can be inorganic also (e.g., —O—Si—O—, or O—P—O—). Additionally, they can be heterogeneous in nature (e.g., composed of organic and inorganic elements). Essentially, any molecule having the appropriate size restrictions and capable of being linked to the various components such as fluorophore and probe can be used as a linker. Examples include the E linker (which also functions as a solubility enhancer), the X linker which is similar to the E linker, the O linker which is a glycol linker, and the P linker which includes a primary aromatic amino group (all supplied by Boston Probes, Inc., now Applied Biosystems). Other suitable linkers are acetyl linkers, 4-aminobenzoic acid containing linkers, Fmoc linkers, 4-aminobenzoic acid linkers, 8-amino-3,6-dioxactanoic acid linkers, succinimidyl maleimidyl methyl cyclohexane carboxylate linkers, succinyl linkers, and the like. Another example of a suitable linker is that described by Haralambidis et al. in U.S. Pat. No. 5,525,465, issued on Jun. 11, 1996. The length of the spacer can vary depending upon the application and the nature of the components being conjugated

The linker molecules may be homo-bifunctional or hetero-bifunctional cross-linkers, depending upon the nature of the molecules to be conjugated. Homo-bifunctional cross-linkers have two identical reactive groups. Hetero-bifunctional cross-linkers are defined as having two different reactive groups that allow for sequential conjugation reaction. Various types of commercially available cross-linkers are reactive with one or more of the following groups: primary amines, secondary amines, sulphydryls, carboxyls, carbonyls and carbohydrates. Examples of amine-specific cross-linkers are bis(sulfosuccinimidyl)suberate, bis[2-(succinimidooxycarbonyloxy)ethyl]sulfone, disuccinimidyl suberate, disuccinimidyl tartarate, dimethyl adipimate.2 HCl, dimethyl pimelimidate.2 HCl, dimethyl suberimidate.2 HCl, and ethylene glycolbis-[succinimidyl-[succinate]]. Cross-linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Cross-linkers preferentially reactive with carbohydrates include azidobenzoyl hydrazine. Cross-linkers preferentially reactive with carboxyl groups include 4-[p-azidosalicylamido]butylamine. Heterobifunctional cross-linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional cross-linkers that react with carbohydrates and sulfhydryls include 4-[N-maleimidomethyl]-cyclohexane-1-carboxylhydrazide.2 HCl, 4-(4-N-maleimidophenyl)-butyric acid hydrazide.2 HCl, and 3-[2-pyridyldithio]propionyl hydrazide. The cross-linkers are bis-[β-4-azidosalicylamido)ethyl]disulfide and glutaraldehyde.

Amine or thiol groups may be added at any nucleotide of a synthetic nucleic acid so as to provide a point of attachment for a bifunctional cross-linker molecule. The nucleic acid may be synthesized incorporating conjugation-competent reagents such as Uni-Link AminoModifier, 3′-DMT-C6-Amine-ON CPG, AminoModifier II, N-TFA-C6-AminoModifier, C6-ThiolModifier, C6-Disulfide Phosphoramidite and C6-Disulfide CPG (Clontech, Palo Alto, Calif.).

In some instances, it may be desirable to use a linker or spacer comprising a bond that is cleavable under certain conditions. For example, the bond can be one that cleaves under normal physiological conditions or that can be caused to cleave specifically upon application of a stimulus such as light, whereby the conjugated entity is released leaving its conjugation partner intact. Readily cleavable bonds include readily hydrolyzable bonds, for example, ester bonds, amide bonds and Schiff's base-type bonds. Bonds which are cleavable by light are known in the art.

Solid Supports or Surfaces

As used herein, a “substrate” can be any substrate on which one or more capture nucleic acids can be immobilized. Examples of substrates that can be used in the compositions and methods provided herein include, for example, include glass, silicon oxides, plastics or metals. Plastic substrates include, for example, acrylonitrile butadiene styrene, polyamide (Nylon), polyamide, polybutadiene, Polybutylene terephthalate, Polycarbonates, poly(ether sulphone) (PES, PES/PEES), poly(ether ether ketone)s, polyethylene (or polyethene), polyethylene glycol, polyethylene oxide, polyethylene terephthalate (PET, PETE, PETP), polyimide, polypropylene, polytetrafluoroethylene (Teflon) perfluoroalkoxy polymer resin (PFA), polystyrene, styrene acrylonitrile, poly(trimethylene terephthalate) (PTT), polyurethane (PU), polyvinylchloride (PVC), polyvinyldifluorine (PVDF), poly(vinyl pyrrolidone) (PVP), Kynar, Mylar, Rilsan, (e.g. polyamide 11 & 12), Ultem, Vectran, Viton, and Zylon. Substrates further include but are not limited to membranes, e.g., natural and modified celluloses such as nitrocellulose or nylon, Sepharose, Agarose, polystyrene, polypropylene, polyethylene, dextran, amylases, polyacrylamides, polyvinylidene difluoride, PEGylated or calcium alginate spheres, other agaroses, and magnetite, including magnetic beads. Substrates also include coblock polymers, which have both hydrophilic and hydrophobic components.

Nucleic acid microarray technology, which is also known by other names including DNA chip technology, gene chip technology, and solid-phase nucleic acid array technology, is well known to those of ordinary skill in the art. Many components and techniques utilized in nucleic acid microarray technology are presented in The Chipping Forecast, Nature Genetics, Vol. 21, January 1999, the entire contents of which is incorporated by reference herein.

Nucleic acid microarray substrates may include but are not limited to glass, silica, aluminosilicates, borosilicates, metal oxides such as alumina and nickel oxide, various clays, nitrocellulose, or nylon. Capture nucleic acids may range in length from 5 to 25 nucleotides, although other lengths may be used. Appropriate capture nucleic acid length may be determined by one of ordinary skill in the art by following art-known procedures.

In one embodiment, the microarray substrate may be coated with a compound to enhance synthesis of the capture nucleic acid on the substrate. Such compounds include, but are not limited to, oligoethylene glycols. In another embodiment, coupling agents or groups on the substrate can be used to covalently link the first nucleotide or oligonucleotide to the substrate. These agents or groups may include, for example, amino, hydroxy, bromo, and carboxy groups. These reactive groups are preferably attached to the substrate through for example an alkylene or phenylene divalent radical, one valence position occupied by the chain bonding and the remaining attached to the reactive groups. These groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, capture nucleic acids are synthesized directly on the substrate in a predetermined grid pattern using methods such as light-directed chemical synthesis, photochemical deprotection, or delivery of nucleotide precursors to the substrate and subsequent capture probe synthesis.

In another embodiment, the substrate may be coated with a compound to enhance binding of the capture probe to the substrate. Such compounds include, but are not limited to: polylysine, amino silanes, amino-reactive silanes, or chromium. In one embodiment, presynthesized capture probes are applied to the substrate in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with methods that include, but are not limited to, UV irradiation. In another embodiment probes are linked to the substrate with heat.

In embodiments of the invention one or more control capture probes are attached to the substrate. Preferably, control probes allow determination of factors such as miRNA quality and binding characteristics, reagent quality and effectiveness, hybridization success, and analysis thresholds and success.

Detection Systems

The miRNA and the solid support upon which it is bound may be analyzed using a detection system capable of holding and moving the solid support in order to analyze signals from various regions of the support. An example of such a device is the Trilogy™ technology developed by U.S. Genomics, Inc. (Woburn, Mass.). This technology is based on earlier Gene Engine™ technology also developed by U.S. Genomics, Inc. Gene Engine™ technology is described in PCT patent applications WO98/35012 and WO00/09757, published on Aug. 13, 1998, and Feb. 24, 2000, respectively, and in issued U.S. Pat. No. 6,355,420 B1, issued Mar. 12, 2002, the contents of which are incorporated by reference herein in their entirety. This system exposes the solid support to an energy source such as optical radiation of a set wavelength and detects signals therefrom. The mechanism for signal emission and detection will depend on the type of label sought to be detected, as described herein.

The Trilogy™ system is a single molecule confocal fluorescence detection platform. The platform enables four-color fluorescent detection in a microfluidic flow stream with engineering modifications to automate sample handling and delivery. In this embodiment, photons emitted by the fluorescently tagged molecules pass through the dichroic mirror and are band-pass filtered to remove stray laser light and any Rayleigh or Raman scattered light. The emission is focused and filtered through 100 micrometer pinholes of multi-mode fiber optic cables coupled to single photon counting modules. A high-speed data acquisition card is used to store photon counts from each channel using a 10 kHz sampling rate. It should be noted that this system has single fluorophore detection sensitivity of four spectrally distinct fluorophores. The Trilogy™ provides real-time counting of individually labeled molecules in a nanoliter interrogation zone. The system detects labeled molecules at low femtomolar concentrations and displays a dynamic range over 4+ logs. The system can accommodate standard sample carriers such as but not limited to 96 well plates or microcentrifuge (e.g., Eppendorf) tubes. The sample volumes may be on the order of microliters (e.g., 1 ul volume).

Trilogy™ is capable of analyzing individual tailed miRNA since it is capable of functioning as a single molecule analysis system. A single molecule analysis system is capable of analyzing single, preferably intact, molecules separately from other molecules. Such a system is sufficiently sensitive to detect signals emitting from a single molecule and its bound probes. Trilogy™ can also function as a linear molecule analysis system in which single molecules are analyzed in a linear manner (i.e., starting at a point along the polymer length and then moving progressively in one direction or another). The methods described herein do not require linear analysis of tailed miRNA which can be analyzed in their entirety.

The Gene Engine™ is also a single molecule analysis system. It allows single polymers to be passed through an interaction station, whereby the units of the polymer or labels of the compound are interrogated individually in order to determine whether there is a detectable label conjugated to the target. Interrogation involves exposing the label to an energy source such as optical radiation of a set wavelength. In response to the energy source exposure, the detectable label emits a detectable signal. The mechanism for signal emission and detection will depend on the type of label sought to be detected.

The systems described herein will encompass at least one detection system. The nature of such detection systems will depend upon the nature of the detectable label. The detection system can be selected from any number of detection systems known in the art. These include an electron spin resonance (ESR) detection system, a charge coupled device (CCD) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

The present invention is further illustrated by the following Examples, which in no way should be construed as further limiting.

### EXAMPLES

### Example 1

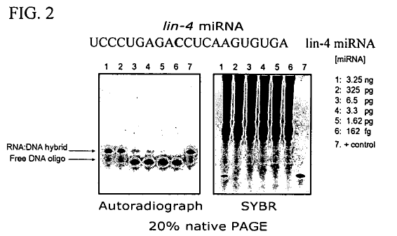

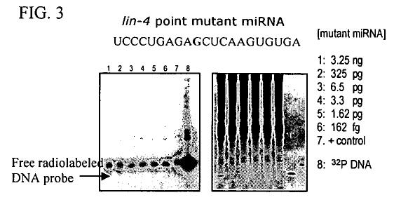

An RNA oligonucleotide identical in sequence to the lin-4 miRNA was titrated in increasing concentrations into 2 micrograms of E. coli total RNA. A radiolabeled DNA oligonucleotide complementary in sequence to lin-4 but containing 10 extra nucleotide bases at its 5′ end was hybridized in solution to the lin-4 spiked NR solutions. When hybridized, this DNA oligomer will generate a 10 base 5′ overhang on the DNA/RNA duplex. In FIG. 2, the left gel shows the resulting autoradiograph. Specific hybridization of lin-4 to the radiolabeled DNA oligonucleotide probe was observed. Lane 7 is a positive control in which a small amount of radiolabeled DNA oligomer was hybridized to several fold molar excess of lin-4 to ensure complete hybridization of radiolabeled oligomer to target miRNA. Sybr Gold staining of the same gel shows the degree of background RNA present in the hybridization reactions. The process was repeated using a lin-4 point mutant as the target miRNA. There was no measurable hybridization of the radiolabeled oligonucleotide to the point mutant miRNA. Similarly, there was no non-specific hybridization to total RNA. The higher molecular weight radiolabeled bands on the gel are the results of radiolabeled branch products that were generated during the synthesis of the DNA oligonucleotide, as is apparent when a high concentration of the radiolabeled DNA oligonucleotide is loaded alone. (FIG. 3, lane 8.)

The long overhang generated when the DNA oligonucleotide hybridizes to the miRNA is used as a template for the primer extension reaction. This reaction uses the miRNA as a primer. In this way, a nucleic acid tail of known sequence can be added to each miRNA. It is will be clear that the system can be designed such that every miRNA has its own specific tail.

### Example 2

The ability of a DNA polymerase to extend off an RNA primer is a vital biological process. The replication of lagging strand requires DNA pol I extension off of short RNA primers. The invention takes advantage of this fundamental process to add capture tails to miRNAs. Several commercially available polymerases are able to extend off the miRNA primers, however they vary in their extension efficiencies. The experiments reported herein used a commercially available thermophilic exopolymerase (i.e., Therminator, New England BioLabs).

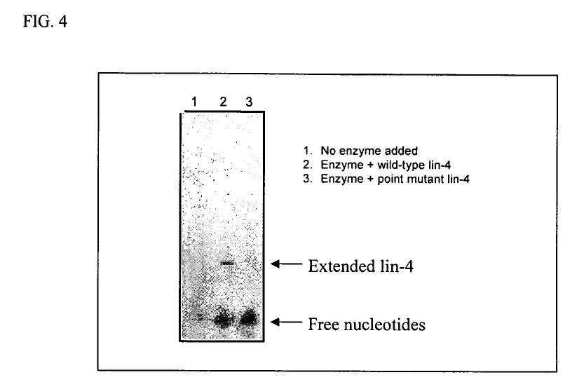

The miRNA targets are not being amplified. Therefore, it is possible to drive the extension reactions to completion with only a limited number of templates. To ensure that miRNA were being specifically extended, extension reactions were conducted using fluorescently labeled nucleotides. Extension reactions used Therminator (New England Biolabs) with sub-optimal concentrations of nucleotides (200 nM). The reactions were cycled (90° C. denaturation, 50° C. hybridization, 70° C. extension) twenty times. The gel in FIG. 4 shows the results of extension reactions conducted on both wild-type lin-4 and the point mutant lin-4. A fluorescently labeled product indicates nucleic acid tail synthesis or polymerization at the 3′ end of the miRNA. Lane 1 represents the reaction without added enzyme. Lane 2 represents the reaction with added enzyme and wild-type lin-4. Lane 3 represents the reaction with added enzyme and point mutant lin-4. Only lane 2 contains the extended lin-4 product. The reaction can be adapted to generate a non-fluorescently labeled unique nucleic acid tail for each miRNA.

### Example 3

The process also involves hybridizing two distinctly labeled probes to the miRNA. This may be done either before or after the tailed miRNA is captured onto a solid support or surface. (See Example 4.) As an example, the probes may be distinct fluorescently labeled probes, 10 nucleotides in length and composed of LNA/DNA elements (i.e., LNA/DNA chimeric probes). In some embodiments, the LNA/DNA chimeric probes offer some advantage over standard DNA oligonucleotide probes. For example, they can off-compete hybridized DNA or RNA probes and they form thermally stable duplexes. This thermal stability ensures complete hybridization will be retained at room temperature and enables hybridization reactions to be carried out at higher temperatures thereby improving hybridization specificity.

### Example 4

The process further involves capture of the tailed miRNA to a solid support or surface. The unique sequence of the nucleic acid tails on the miRNA are hybridized to complementary capture nucleic acids positioned on pre-determined 2-dimensional locations on a surface or support such as a silica chip. LNA capture probes are immobilized on for example silica chips. (Tolstrup N. et al. 1993.) Linkers or spacers can be used to position the capture probes away from the solid surfaces in order to minimize steric hindrance the might interfere with hybridization of the capture probe to the tailed miRNA. An example of a linker or spacer is a 3′-ethylene glycol scaffold. If the tailed miRNA has already been hybridized to the probes of Example 3, then the capture hybridization is carried out under conditions that do not cause denaturation of the probes from the miRNA. Moreover, shorter capture probes are possible by incorporating LNA elements into the probes.

### Example 5

The final step in the process involves measuring signal from the captured miRNAs. A single molecule detection platform can be used to scan the surface of the solid support (e.g., the silica chip surface) and thereby quantitate the amount of signal from each pre-determined region. It is possible that up to 5,000 different miRNA may be analyzed per day using automated detection systems. For example, the Trilogy™ analysis system may be used and/or adapted for this purpose. In one embodiment, the instrument's confocal microscopy arrangement may be replaced with a linear array, single electron multiplying CCD (EM-CCD). EM-CCDs are an emerging technology with on-chip signal amplification that essentially eliminates the largest source of noise in conventional CCDs (i.e., the read-out noise).

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

All references, patents and patent applications that are recited in this application are incorporated by reference herein in their entirety.

What is claimed is:

1. A method for quantitating microRNA in a sample comprising
contacting a template nucleic acid with a microRNA and allowing the template nucleic acid to bind to the microRNA thereby creating a 5′ template overhang,
polymerizing a nucleic acid tail onto the microRNA wherein the nucleic acid tail is complementary to the 5′ template overhang and thereby creating a tailed microRNA,
separating the template nucleic acid from the tailed microRNA,
contacting a first and a second sequence specific probe with the tailed microRNA and allowing the first and second sequence specific probes to bind to the tailed microRNA wherein the first and second sequence specific probes are complementary to the microRNA or the nucleic acid tail,
contacting the tailed microRNA with a solid support conjugated to a nucleic acid complementary to the nucleic acid tail and allowing the tailed microRNA to bind to the solid support at a defined location, and
detecting the level of binding of the tailed microRNA to the solid support based on the presence of the first and second sequence specific probes at the defined location.

2. The method of claim 1, wherein the first and second sequence specific probes are conjugated to first and second detectable labels.

3. The method of claim 2, wherein the first and second detectable labels are first and second fluorophores.

4. The method of claim 3, wherein the first fluorophore is distinct from the second fluorophore.

5. The method of claim 1, wherein the template nucleic acid is about 50% longer than the microRNA.

6. The method of claim 1, wherein the 5′ template overhang is at least 10 bases in length.

7. The method of claim 1, wherein the tailed microRNA is contacted with the first and second sequence specific probes prior to contact with the solid support.

8. The method of claim 1, wherein the tailed microRNA is contacted with the first and second sequence specific probes after contact with and binding to the solid support

9. The method of claim 1, wherein the microRNA is less than 25 nucleotides in length.

10. The method of claim 1, wherein the template nucleic acid is a DNA.

11. The method of claim 1, wherein the first and second sequence specific probes are comprised of LNA or are LNA/DNA chimerae.

12. The method of claim 1, wherein the solid support is a silica chip.

13. The method of claim 1, further comprising quantitating a plurality of microRNA.

14. The method of claim 1, wherein the defined location on the solid support has a plurality of nucleic acids conjugated to it.

15. The method of claim 1, wherein the nucleic acid tail is polymerized by a primer extension reaction.

16. The method of claim 15, wherein the primer extension reaction comprises a thermophilic exopolymerase.

17. The method of claim 1, wherein the nucleic acid tail is fluorescent.

18. The method of claim 1, wherein the nucleic acid complementary to the nucleic acid tail is comprised of an LNA or is an LNA/DNA chimera.

19. The method of claim 1, wherein the nucleic acid complementary to the nucleic acid tail is tethered to the solid support via a 3′ ethylene glycol scaffold.
